(19) United States
(12) Patent Application Publication
(10) Pub. No.: US 2026/0027288 A1
(43) Pub. Date: Jan. 29, 2026

(54) Medical Tube Management Systems and Methods

(71) Applicant: Sage Products LLC, Cary, IL (US)

(72) Inventors: Daniel R. Ulreich, McHenry, IL (US); Emma Kahn, Cary, IL (US); Jenna Krause, Cary, IL (US)

(21) Appl. No.: 18/996,228

(22) PCT Filed: Jul. 17, 2023
(86) PCT No.: PCT/US2023/027906

§ 371 Date: Jan. 17, 2025

Related U.S. Application Data
(60) Provisional application No. 63/389,931, filed on Jul. 17, 2022.

(51) Int. Cl. A61M 5/14

(52) U.S. Cl. CPC A61M 5/1418

(57) ABSTRACT

In an example, a line management system includes a main clip and a plurality of line retention structures. The main clip includes a first lateral wall, a second lateral wall, and an end wall. The first lateral wall extends from a first lateral end of the end wall and the second lateral wall extends from a second lateral end of the end wall such that an interior channel is defined between an inner surface of the first lateral wall, an inner surface of the second lateral wall, and an inner surface of the end wall. The plurality of line retention structures are on an outer surface of the end wall.

### CROSS-REFERENCE TO RELATED APPLICATIONS

This application claims the benefit of priority to U.S. Provisional Application No. 63/389,931, filed Jul. 17, 2022, the contents of which are hereby incorporated by reference in their entirety.

### FIELD

The present disclosure generally relates to medical tube management systems and methods and, in particular, medical tube management systems and methods for organizing and coupling a plurality of fluid tubes to a support structure.

### BACKGROUND

During some medical procedures, a patient may be infused with a plurality of fluids such as, for example, saline, blood, and/or medications. In general, the fluids are provided to the patient through a plurality of tubes such as, for example, intravenous (IV) lines. Given the number of tubes and/or the number of other cables and cords in a healthcare setting, these tubes may present various challenges. For example, the tubes may become tangled. This can make it difficult to determine which tube is being used for which fluid. Additionally, tangled tubes may lead to greater tension being applied to the patient when, for instance, a patient moves. In some instances, this may even lead to a tube inadvertently decoupling from the patient. Another problem may be that tangled tubes may become kinked, which may impair the flow of fluid to the patient. Yet another problem may be that the tubes may present a tripping hazard for medical personnel.

### SUMMARY

In an example, a line management system is described. The line management system includes a main body, an attachment mechanism, and a first end cap. The main body includes a first end surface, a second end surface opposite the first end surface, a first major surface extending between the first end surface and the second end surface, and a second major surface extending between the first end surface and the second end surface. The first major surface includes a plurality of first line retention structures that each extend between the first end surface and the second end surface. The second major surface is opposite the first major surface. The attachment mechanism is configured to couple the main body to a support structure. The first end cap removably coupled to the first end surface, wherein the first end cap comprises a plurality of second line retention structures. When the first end cap is coupled to the first end surface of the main body, each second line retention structure is aligned with a corresponding one of the plurality of first line retention structures such that the second line retention structure and the corresponding one of the plurality of first line retention structures are configured to simultaneously retain to a respective fluid tube of a plurality of fluid tubes.

In another example, a line management system includes a main clip and a plurality of line retention structures. The main clip includes a first lateral wall, a second lateral wall, and an end wall. The first lateral wall extends from a first lateral end of the end wall and the second lateral wall extends from a second lateral end of the end wall such that an interior channel is defined between an inner surface of the first lateral wall, an inner surface of the second lateral wall, and an inner surface of the end wall. The plurality of line retention structures are on an outer surface of the end wall.

The features, functions, and advantages that have been discussed can be achieved independently in various embodiments or may be combined in yet other embodiments further details of which can be seen with reference to the following description and drawings.

### BRIEF DESCRIPTION OF THE FIGURES

The novel features believed characteristic of the illustrative embodiments are set forth in the appended claims. The illustrative embodiments, however, as well as a preferred mode of use, further objectives and descriptions thereof, will best be understood by reference to the following detailed description of an illustrative embodiment of the present disclosure when read in conjunction with the accompanying drawings, wherein:

FIG. 1A depicts an exploded view of the line management system, according to an example.

FIG. 1B depicts a perspective view of a main body of the line management system depicted in FIG. 1A, according to an example.

FIG. 1C depicts a first view of the line management system of FIG. 1A coupled to a support structure and a plurality of fluid tubes, according to an example.

FIG. 1D depicts a second view of the line management system of FIG. 1A coupled to a support structure and a plurality of fluid tubes, according to an example.

FIG. 1E depicts an end cap of the line management system of FIG. 1A coupled to the fluid tubes, according to an example.

FIG. 1F depicts a third view of the line management system of FIG. 1A coupled to the support structure and the fluid tubes, according to an example.

FIG. 2A depicts a perspective view of the line management system coupled to a support structure and a plurality of fluid lines, according to an example.

FIG. 2B depicts a perspective view of a main clip of the line management system of FIG. 2A, according to an example.

FIG. 2C depicts a side view of the main clip of FIG. 2B, according to an example.

FIG. 3A depicts the line management system coupled to a support structure 102, according to an example.

FIG. 3B depicts a perspective view of a main clip of the line management system shown in FIG. 3A, according to an example.

FIG. 3C depicts a side view of the main clip of FIG. 3B, according to an example.

FIG. 4A depicts the line management system coupled to a support structure and a plurality of fluid tubes, according to another example.

FIG. 4B depicts a main clip and a secondary clip of the line management system shown in FIG. 4A, according to an example.

FIG. 5A depicts a line management system coupled to a first support structure, according to another example.

FIG. 5B depicts the line management system of FIG. 5A coupled to a second support structure and a plurality of fluid tubes, according to an example.

FIG. 5C depicts a perspective view of the main clip, a secondary clip, and a slack holder for the line management system of FIG. 5A, according to an example.

FIG. 5D depicts a side view of the main clip, the secondary clip, and the slack holder for the line management system of FIG. 5A, according to an example.

FIG. 6 depicts a perspective view of a line management system coupled to a support structure and a plurality of fluid tubes 104, according to another example.

FIG. 7A depicts a perspective view the line management system, according to another example.

FIG. 7B depicts a side view of the line management system shown in FIG. 7A, according to an example.

FIG. 7C depicts a top view of the line management system shown in FIG. 7A, according to an example.

FIG. 8A depicts a perspective view the line management system, according to another example.

FIG. 8B depicts a side view of the line management system shown in FIG. 8A, according to an example.

FIG. 9 depicts a flow chart for a process of using a line management system, according to an example.

FIG. 10 depicts a flow chart for a process of using a line management system that can be used with at least the process shown in FIG. 9, according to an example.

FIG. 11 depicts a flow chart for a process of using a line management system that can be used with at least the process shown in FIG. 9, according to an example.

FIG. 12 depicts a flow chart for a process of using a line management system that can be used with at least the process shown in FIG. 11, according to an example.

FIG. 13 depicts a flow chart for a process of using a line management system that can be used with at least the process shown in FIG. 12, according to an example.

FIG. 14 depicts a flow chart for a process of using a line management system, according to another example.

FIG. 15 depicts a flow chart for a process of using a line management system that can be used with at least the process shown in FIG. 14, according to an example.

FIG. 16 depicts a flow chart for a process of using a line management system that can be used with at least the process shown in FIG. 14, according to an example.

FIG. 17 depicts a flow chart for a process of using a line management system that can be used with at least the process shown in FIG. 14, according to an example.

FIG. 18 depicts a flow chart for a process of using a line management system that can be used with at least the process shown in FIG. 14, according to an example.

### DETAILED DESCRIPTION

Disclosed embodiments will now be described more fully hereinafter with reference to the accompanying drawings, in which some, but not all of the disclosed embodiments are shown. Indeed, several different embodiments may be described and should not be construed as limited to the embodiments set forth herein. Rather, these embodiments are described so that this disclosure will be thorough and complete and will fully convey the scope of the disclosure to those skilled in the art.

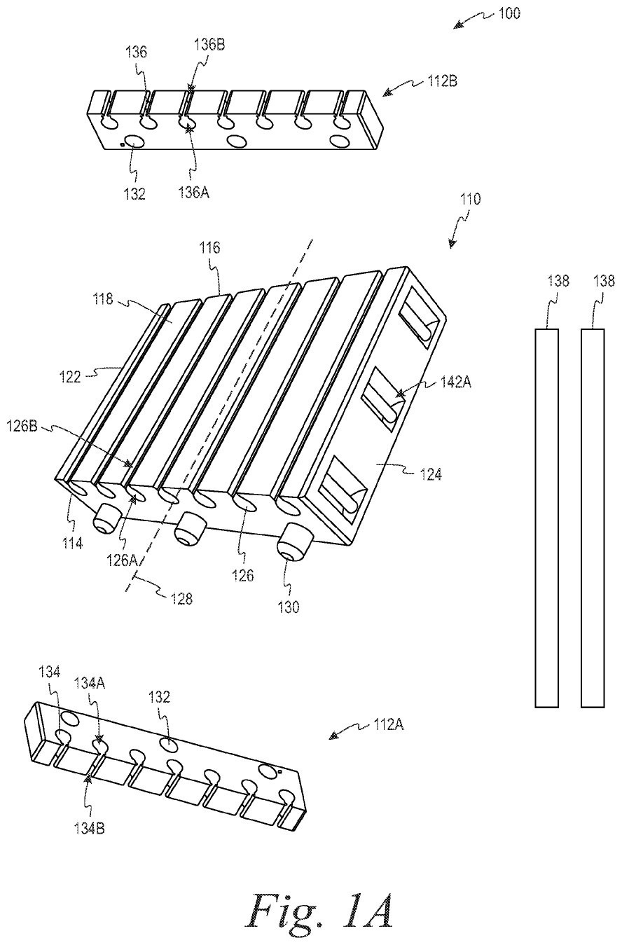

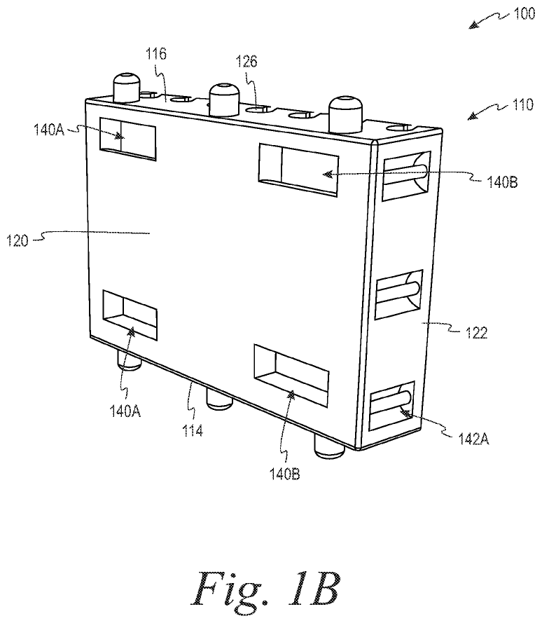

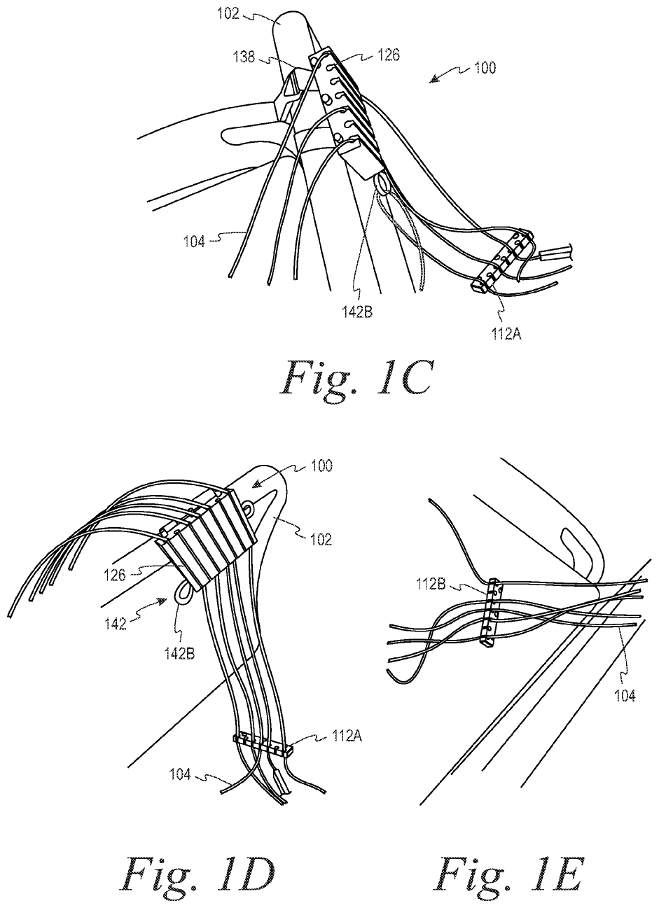

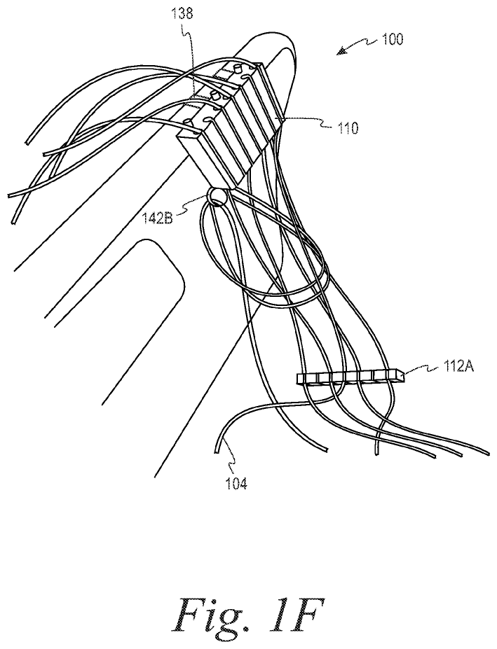

Referring now to FIGS. 1A-1F, a line management system 100 is illustrated according to an example. More specifically, FIG. 1A depicts an exploded view of the line management system 100, FIG. 1B depicts a perspective view of the line management system 100, FIG. 1C depicts a first view of the line management system 100 coupled to a support structure 102 and with a plurality of fluid tubes 104 coupled to the line management system 100, FIG. 1D depicts a second view of the line management system 100 coupled to the support structure 102 and the fluid tubes 104, FIG. 1E depicts an end cap of the line management system 100 coupled with the fluid tubes 104, and FIG. 1F depicts a third view of the line management system coupled to the support structure 102 and the fluid tubes 104, according to an example.

As shown in FIG. 1A, the line management system 100 includes a main body 110 and at least one end cap 112A, 112B. The main body 110 includes a first end surface 114, a second end surface 116 opposite the first end surface 114, a first major surface 118 extending between the first end surface 114 and the second end surface 116, and a second major surface 120 extending between the first end surface 114 and the second end surface 116. The second major surface 120 is opposite the first major surface 118.

The main body 110 can also include a first lateral surface 122 and a second lateral surface 124. The first lateral surface 122 extends between the first major surface 118, the second major surface 120, the first end surface 114, and the second end surface 116. The second lateral surface 124 is opposite the first lateral surface 122, and extends between the first major surface 118, the second major surface 120, the first end surface 114, and the second end surface 116.

The first major surface 118 includes a plurality of first line retention structures 126 that each extend between the first end surface 114 and the second end surface 116. For instance, as shown in FIGS. 1A and 1C-1F, the main body 110 can have a longitudinal axis 128 that extends between the first end surface 114 and the second end surface 116, and each first line retention structure 126 can include a channel 126A defined by the main body 110 and having a longitudinal axis that is parallel to the longitudinal axis 128 of the main body 110. Additionally, the channel 126A of each first line retention structure 126 can include an aperture 126B that (i) extends along a length of the channel 126A and (ii) has a width that is smaller than a diameter of the channel 126A. The aperture 126B of each first line retention structure 126 can additionally or alternatively have a width that is smaller than a diameter of the fluid tube 104 that is coupled to the first line retention structure 126, whereas the channel 126A of the first line retention structure 126 can have a diameter that is greater than a diameter of the fluid tube 104. This can help to retain the fluid tube 104 in the first line retention structure 126. In one example, the width of each aperture 126B can be approximately 3 millimeters (mm) and the diameter of each channel 126A can be approximately 7 mm. This can help to couple and retain the fluid lines in the form of IV lines.

Within examples, at least a portion of the main body 110 that defines the first line retention structures 126 can be formed from an elastically deformable material. In this arrangement, a plurality of fluid tubes 104 can be inserted into the channels 126A through the apertures 126B of the first line retention structures 126. This can allow the fluid tubes 104 to be coupled to the main body 110 while the ends of the fluid tubes 104 are coupled to the patient and/or a fluid source. FIGS. 1C-1D depict the fluid tubes 104 received in the channels 126A and the first line retention structures 126 retaining the fluid tubes 104 in the channels 126A.

As shown in FIG. 1A, the at least one end cap 112A, 112B includes a first end cap 112A that is configured to be removably coupled to the first end surface 114 of the main body 110. In the example shown in FIGS. 1A-1F, (i) one of the first end surface 114 of the main body 110 or the first end cap 112A can include one or more protrusions 130, (ii) another of the first end surface 114 or the first end cap 112A can include one or more receptacles 132, and (iii) the first end cap 112A can be removably coupled to the first end surface 114 of the main body 110 by a press-fit coupling between the one or more protrusions 130 and the one or more receptacles 132. In other examples, the first end cap 112A can be removably coupled to the first end surface 114 by, for instance, a snap/clip coupling, a hook-and-loop coupling, a magnetic coupling, and/or an adhesive.

The first end cap 112A includes a plurality of second line retention structures 134 that are configured to couple to the fluid tubes 104. When the first end cap 112A is coupled to the first end surface 114 of the main body 110, each second line retention structure 134 can be aligned with a corresponding one of the first line retention structures 126 such that the second line retention structure 134 and the corresponding one of the first line retention structures 126 are configured to simultaneously retain to a respective fluid tube 104 of a plurality of fluid tubes 104. For example, each second line retention structure 134 can include a second channel 134A defined by the first end cap 112A, and the second channel 134A of each second line retention structure 134 can include an aperture 134B that (i) extends along a length of the second channel 134A and (ii) has a width that is smaller than a diameter of the second channel 134A. Additionally, the second channel 134A of each second line retention structure 134 can include an aperture 134B that (i) extends along a length of the second channel 134A and (ii) has a width that is smaller than a diameter of the second channel 134A. Each second line retention structure 134 can have a longitudinal axis that is parallel to the longitudinal axis 128 of the main body 110. The aperture 134B of each second line retention structure 134 can additionally or alternatively have a width that is smaller than a diameter of the fluid tube 104 that is coupled to the second line retention structure 134, whereas the second channel 134A of the second line retention structure 134 can have a diameter that is greater than a diameter of the fluid tube 104. This can help to retain the fluid tube 104 in the second line retention structure 134.

In some examples, the line management system 100 can also include a second end cap 112B that can be removably coupled to the second end surface 116 of the main body 110. In the example shown in FIGS. 1A-1F, (i) one of the second end surface 116 of the main body 110 or the second end cap 112B can include the one or more protrusions 130, (ii) another of the second end surface 116 or the second end cap 112B can include the one or more receptacles 132, and (iii) the second end cap 112B can be removably coupled to the second end surface 116 of the main body 110 by a press-fit coupling between the one or more protrusions 130 and the one or more receptacles 132. In other examples, the second end cap 112B can be removably coupled to the second end surface 116 by, for instance, a snap/clip coupling, a hook-and-loop coupling, a magnetic coupling, and/or an adhesive.

The second end cap 112B includes a plurality of third line retention structures 136 that are configured to couple to the fluid tubes 104. When the second end cap 112B is coupled to the second end surface 116 of the main body 110, each third line retention structure 136 can be aligned with a corresponding one of the first line retention structures 126 such that the third line retention structure 136 and the corresponding one of the first line retention structures 126 are configured to simultaneously retain to a respective fluid tube 104 of a plurality of fluid tubes 104. For example, each third line retention structure 136 can include a third channel 136A defined by the second end cap 112B, and the third channel 136A of each third line retention structure 136 can include an aperture 136B that (i) extends along a length of the third channel 136A and (ii) has a width that is smaller than a diameter of the third channel 136A. Additionally, the third channel 136A of each third line retention structure 136 can include an aperture 136B that (i) extends along a length of the third channel 136A and (ii) has a width that is smaller than a diameter of the third channel 136A. Each third line retention structure 136 can have a longitudinal axis that is parallel to the longitudinal axis 128 of the main body 110. The aperture 136B of each third line retention structure 136 can additionally or alternatively have a width that is smaller than a diameter of the fluid tube 104 that is coupled to the third line retention structure 136, whereas the third channel 136A of the third line retention structure 136 can have a diameter that is greater than a diameter of the fluid tube 104. This can help to retain the fluid tube 104 in the third line retention structure 136.

Within examples, at least a portion of the first end cap 112A and/or the second end cap 112B that defines the second line retention structures 134 and/or the third line retention structures 136, respectively, can be formed from an elastically deformable material. In this arrangement, the fluid tubes 104 can be inserted into the second channels 134A through the apertures 134B of the second line retention structures 134 and the fluid tubes 104 can be inserted into the third channels 136A through the apertures of the third line retention structures 136. This can allow the fluid tubes 104 to be coupled to the first end cap 112A and/or the second end cap 112B while the ends of the fluid tubes 104 are coupled to the patient and/or a fluid source. FIGS. 1C-1D depict the fluid tubes 104 received in the channels 126A and the first line retention structures 126 retaining the fluid tubes 104 in the channels 126A.

In some examples, the widths of each aperture 134B, 136B of the second line retention structures 134 and the third line retention structures 136 can be equal to the width of the apertures 126B of the first line retention structures 126. In one example, the width of each aperture 134B, 136B can be approximately 3 millimeters (mm) and the diameter of each channel 126A can be approximately 7 mm. This can help to couple and retain the fluid lines in the form of IV lines.

As shown in FIGS. 1A and 1C, the line management system 100 can also include an attachment mechanism 138 that is configured to couple the main body 110 to the support structure 102. As examples, the support structure 102 can be a hospital bed, a wheel chair, and/or an IV pole. Also, as examples, the attachment mechanism 138 at least one device selected from a group consisting of: one or more straps, one or more clips, and one or more adhesives.

In the example shown in FIGS. 1A and 1C, the attachment mechanism 138 includes one or more straps. Additionally, as shown in FIG. 1B, the main body 110 can include one or more strap slots 140A, 140B that are each configured to retain the one or more straps. For instance, as shown in FIG. 1B, the one or more strap slots 140A, 140B can include a first strap slot 140A and a second strap slot 140B. The first strap slot 140A and the second strap slot 140B can each extend in a direction between the first end surface 114 and the second end surface 116. In FIG. 1B, the first strap slot 140A and the second strap slot 140B each extend between two respective openings in the second major surface 120.

As shown in FIG. 1C, the straps can extend through the strap slots 140A, 140B and around the support structure 102 to couple the main body 110 to the support structure 102. The straps can include one or more fastening mechanisms such as, for example, one or more buttons, one or more hook-and-loop fasteners, one or more buckles, and/or one or more cinch mechanisms.

As shown in FIGS. 1C-1E, the main body 110 is coupled to the support structure 102 and the fluid tubes 104 are coupled to respective ones of the first line retention structures 126 of the main body 110. This can help to organize the fluid tubes 104 and mitigate tangling and/or kinking.

Additionally, as shown in FIG. 1D, the first end cap 112A can be decoupled from the main body 110 while the fluid tubes 104 are coupled to the second line retention structures 134. This can help to organize the fluid tubes 104 and mitigate tangling and/or kinking between the support structure 102 and one or more fluid sources (not shown) coupled to the fluid tubes 104. Within examples, the second line retention structures 134 can have a size and a shape such that the first end cap 112A is slidable along the fluid tubes 104 while the fluid tubes 104 are coupled to the second line retention structures 134. This can provide for adjusting the position of the first end cap 112A relative to the main body 110 without decoupling the fluid tubes 104 from either the main body 110 or the first end cap 112A.

As shown in FIG. 1E, the second end cap 112B can be decoupled from the main body 110 while the fluid tubes 104 are coupled to the third line retention structures 136. This can help to organize the fluid tubes 104 and mitigate tangling and/or kinking between the support structure 102 and the patient. Within examples, the third line retention structures 136 can have a size and a shape such that the second end cap 112B is slidable along the fluid tubes 104 while the fluid tubes 104 are coupled to the third line retention structures 136. This can provide for adjusting the position of the second end cap 112B relative to the main body 110 without decoupling the fluid tubes 104 from either the main body 110 or the second end cap 112B.

As shown in FIGS. 1A-1B, 1D and IF, the line management system 100 can also include a slack holder 142 that can couple to and retain one or more of the fluid tubes 104 while the fluid tubes 104 are also coupled to the first line retention structures 126, the second line retention structures 134, and/or the third line retention structures 136. In this example, the slack holder 142 can include (i) one or more slack slots 142A on the first lateral surface 122 and/or the second lateral surface 124 of the main body 110 and (ii) one or more slack straps 142B, which are each coupled to a respective one of the slack slots 142A. The slack strap(s) 142B can be in the form of a loop. The slack strap(s) 142B can include a fastener (e.g., a buckle, a button, a hook-and-loop fastener) to provide for opening and closing the loop of the slack strap(s) 142B and/or coupling and decoupling the slack strap(s) 142B from the slack slot(s) 142A.

In this arrangement, an excess portion of one or more the fluid tubes 104 can be formed into one or more loops of tubing and coupled to the main body 110 by the slack strap(s) 142B of the slack holder 142 as shown in FIG. 1F. This can further help to mitigate tangling and/or kinking.

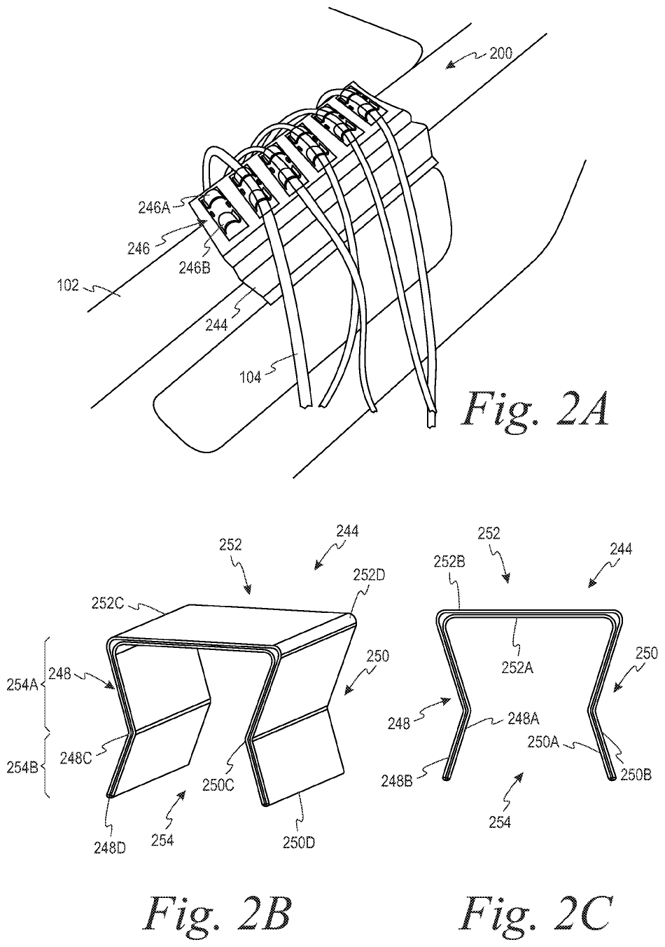

Referring now to FIGS. 2A-2C, a line management system 200 is shown according to another example. FIG. 2A depicts a perspective view of the line management system 200 coupled to a support structure 102 and a plurality of fluid tubes 104, FIG. 2B depicts a perspective view of a main clip 244 of the line management system 200, and FIG. 2C depicts a side view of the main clip 244, according to an example.

As shown in FIGS. 2A-2C, the line management system 200 includes the main clip 244 and a plurality of line retention structures 246. The main clip 244 includes a first lateral wall 248, a second lateral wall 250, and an end wall 252. The main clip 244 also includes an inner surface defined by an inner surface 248A of the first lateral wall 248, an inner surface 250A of the second lateral wall 250, and an inner surface 250A of the end wall 252. The main clip 244 further includes an outer surface defined by an outer surface 248B of the first lateral wall 248, an outer surface 250B of the second lateral wall 250, and an outer surface 250B of the end wall 252.

The first lateral wall 248 extends from a first lateral end 252C of the end wall 252 and the second lateral wall 250 extends from a second lateral end 252D of the end wall 252 such that an interior channel 254 is defined between an inner surface 248A of the first lateral wall 248, an inner surface 250A of the second lateral wall 250, and an inner surface 252A of the end wall 252. In this arrangement, the main clip 244 can receive the support structure 102 in the interior channel 254 to couple the main clip 244 to the support structure 102.

In some examples, the main clip 244 can be formed from an elastically deformable material. This can help to expand a width of the interior channel 254 (e.g., in a dimension extending between the first lateral wall 248 and the second lateral wall 250) to receive a portion of the support structure 102 having a greater width, and apply by the main clip 244 a clamping force to the support structure 102.

In the example shown in FIGS. 2A-2C, the interior channel 254 of the main clip 244 includes: (i) a first portion 254A extending from the end wall 252 to an intermediate point 248C on the first lateral wall 248 and an intermediate point 250C on the second lateral wall 250, and (ii) a second portion 254B extending from the intermediate point 248C on the first lateral wall 248 and the intermediate point 250C on the second lateral wall 250 to a free end 248D of the first lateral wall 248 and a free end 250D of the second lateral wall 250. As shown in FIGS. 2B-2C, the first portion 254A of the interior channel 254 can have a different shape than the second portion 254B of the interior channel 254. For instance, the first portion 254A can have a width that decreases in a direction from the end wall 252 towards the intermediate points 248C, 250C, and the second portion 254B can have a width that increases in a direction from the intermediate points 248C, 250C toward the free end 248D of the first lateral wall 248 and the free end 250D of the second lateral wall 250. This shape can help to enhance the clamping force that the main clip 244 can apply to the support structure 102 when the main clip 244 is coupled to the support structure 102. For instance, when the support structure 102 is initially inserted in the interior channel 254 of the main clip 244, the outwardly flared second portion 254B can assist in expanding the size of the interior channel 254. After the support structure is further inserted into the interior channel 254 past the intermediate points 248C, 250C and into the first portion 254A of the interior channel 254, the inwardly flared shape of the first portion 254A can help to apply the clamping force to the support structure 102.

As shown in FIG. 2A, the plurality of line retention structures 246 are on the outer surface 252B of the end wall 252. Additionally, as shown in FIG. 2A, each line retention structure 246 can couple to a respective one of the fluid tubes 104. In this example, each line retention structure 246 can be configured such that when the fluid tube 104 is coupled to the line retention structure 246, the fluid tube 104 extends through the line retention structure 246 in a lateral direction extending between the first lateral wall 248 and the second lateral wall 250.

In FIG. 2A, each line retention structure 246 includes one or more clips 246A, 246B. For instance, each line retention structure 246 includes a first clip 246A extending in a first direction and a second clip 246B extending in a second direction, which is opposite the first direction. In particular, the first clip 246A extends in the first direction from a fixed end coupled to the outer surface 252B of the end wall 252 to a free end, and the second clip 246B extends in the second direction from a fixed end coupled to the outer surface 252B of the end wall 252 to a free end. In this arrangement, the fluid tube 104 can be inserted in the second direction into the first clip 246A, and inserted in the first direction into the second clip 246B. Arranging the first clip 246A and the second clip 246B in opposite directions can help to mitigate the fluid tubes 104 inadvertently decoupling from the line retention structures 246. However, the one or more clips 246A, 246B can be arranged in the same direction in other examples. This may provide for easier coupling and decoupling of the fluid tubes 104 to the line retention structures 246.

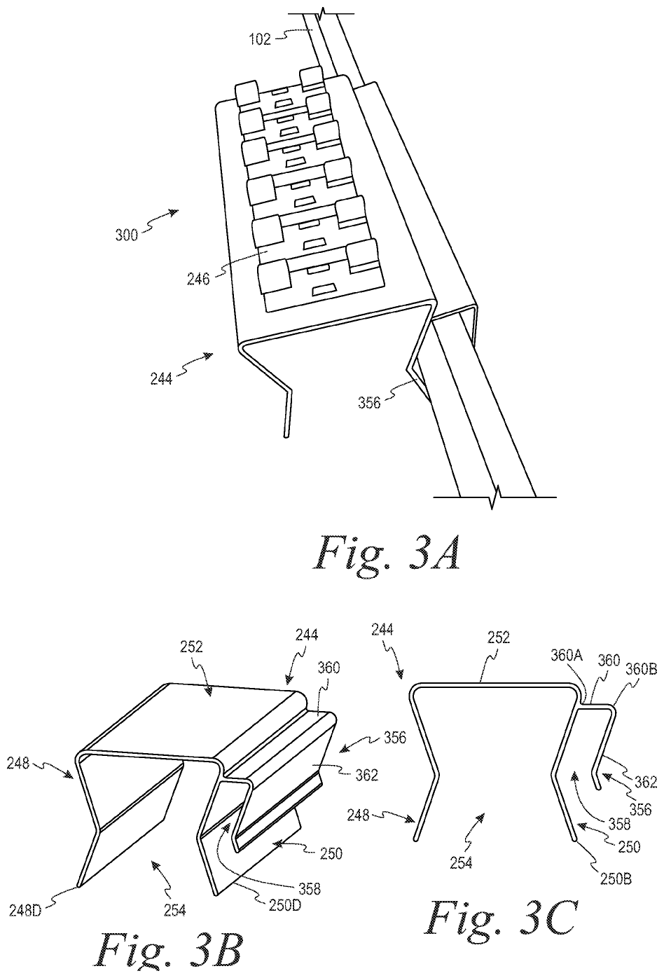

Referring now to FIGS. 3A-3C, a line management system 300 is shown according to another example. FIG. 3A depicts the line management system 300 coupled to a support structure 102, FIG. 3B depicts a perspective view of the main clip 244 and a secondary clip 356 of the line management system 300, and FIG. 3C depicts a side view of the main clip 244 and the secondary clip 356 of FIG. 3B, according to an example.

As shown in FIG. 3A, the line management system 300 of FIGS. 3A-3C is substantially similar or identical to the line management system 200 of FIGS. 2A-2C, except the line management system 300 includes the secondary clip 356 extending from the outer surface 250B of the second lateral wall 250.

The secondary clip 356 can at least partially define a second interior channel 358 that can receive the support structure 102 to couple the line management system 300 to the support structure 102. The second interior channel 358 can have a different shape and/or different dimensions than the interior channel 254 of the main clip 244. This can help to configure the line management system 300 for coupling with a wider variety of support structures 102 (e.g., having different shapes and/or dimensions) than the line management system 200, which omits the secondary clip 356.

As shown in FIGS. 3A-3C, the secondary clip 356 can include a lateral segment 360 and a vertical segment 362. The lateral segment 360 can extend from the second lateral wall 250 (e.g., in a plane that is parallel to or coplanar with a plane in which the end wall 252 extends). The lateral segment 360 can have a first end 360A at the second lateral wall 250 and a second end 360B opposite the first end 3560A. The vertical segment 362 can extend from the second end 360B of the lateral segment 360 such that the second interior channel 358 is defined between the outer surface 25B of the second lateral wall 250, an inner surface of the lateral segment 360, and an inner surface of the vertical segment 362. In this arrangement, the second interior channel 358 can have a longitudinal axis that is approximately parallel to a longitudinal axis of the interior channel 254 of the main clip 244. This can help to orient the fluid tubes 104 in a direction that is transverse (e.g., approximately perpendicular) to an axis of the support structure 102 to which the line management system 300 is coupled.

In some examples, the vertical segment 362 can be parallel to the second lateral wall 250. For instance, in FIGS. 3B-3C, the vertical segment 362 and the lateral segment 360 define an acute angle such that is the same or substantially similar to an acute angle between the end wall 252 and the second lateral wall 250. Additionally, in some examples, the vertical segment 362 can extend past the intermediate point 250C, the vertical segment 362 can be parallel to the second lateral wall 250 on both sides of the intermediate point 250C. In other examples, a portion or an entirety of the vertical segment 362 may not be parallel to the second lateral wall 250.

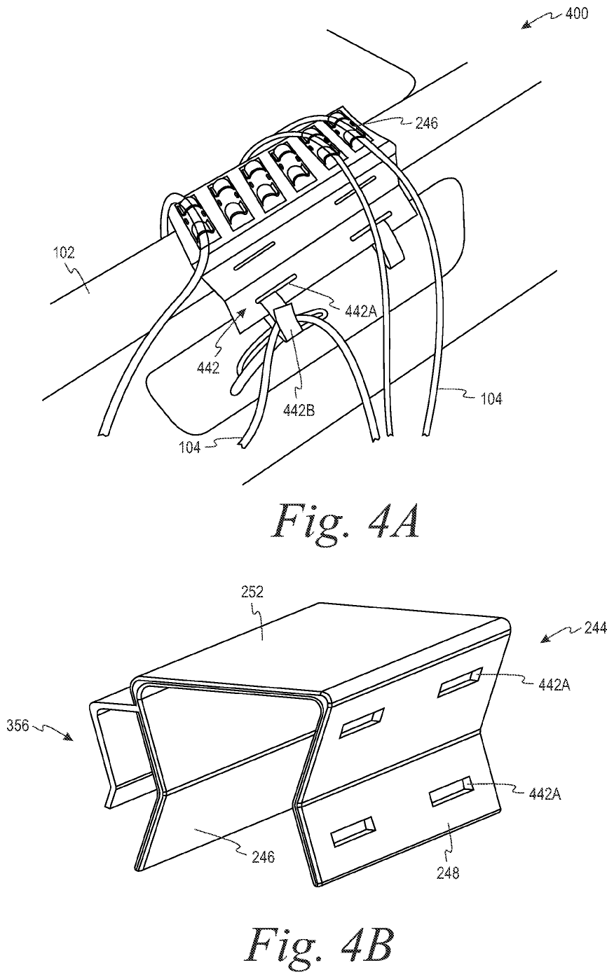

Referring now to FIGS. 4A-4B, a line management system 400 is shown according to another example. FIG. 4A depicts the line management system 400 coupled to a support structure 102 and a plurality of fluid tubes 104, and FIG. 4B depicts the main clip 244 and a secondary clip 356 of the line management system 400, according to an example.

As shown in FIG. 4A, the line management system 400 is substantially similar or identical to the line management system 300, except the line management system 400 includes also includes a slack holder 442. Similar to the slack holder 142 described above, the slack holder 442 can couple to and retain one or more of the fluid tubes 104 while the fluid tubes 104 are also coupled to the line retention structures 246.

In this example, the slack holder 442 can include (i) one or more slack slots 442A on the first lateral wall 248 of the main clip 244 and (ii) one or more slack straps 442B, which are each coupled to a respective one of the slack slots 442A. The slack strap(s) 442B can be in the form of a loop. The slack strap(s) 442B can include a fastener (e.g., a buckle, a button, a hook-and-loop fastener) to provide for opening and closing the loop of the slack strap(s) 442B and/or coupling and decoupling the slack strap(s) 442B from the slack slot(s) 442A.

In this arrangement, an excess portion of one or more the fluid tubes 104 can be formed into one or more loops of tubing and coupled to the main clip 244 by the slack strap(s) 442B of the slack holder 442 as shown in FIG. 2A. This can further help to mitigate tangling and/or kinking.

Although the slack slot(s) 442A are in the first lateral wall 248 in FIGS. 4A-4B, the slack slot(s) 442A can be additionally or alternatively in the second lateral wall 250 in another example. More generally, at least one of the first lateral wall 248 or the second lateral wall 250 can include the plurality of slack slots 442A that are configured to couple to the slack straps 442B.

Additionally, although the line management system 400 includes the secondary clip 356, the line management system 400 can omit the secondary clip 356. For instance, the slack holder 442 of FIGS. 4A-4B can be provided in the line management system 200 shown in FIGS. 2A-2C or any other line management system described herein.

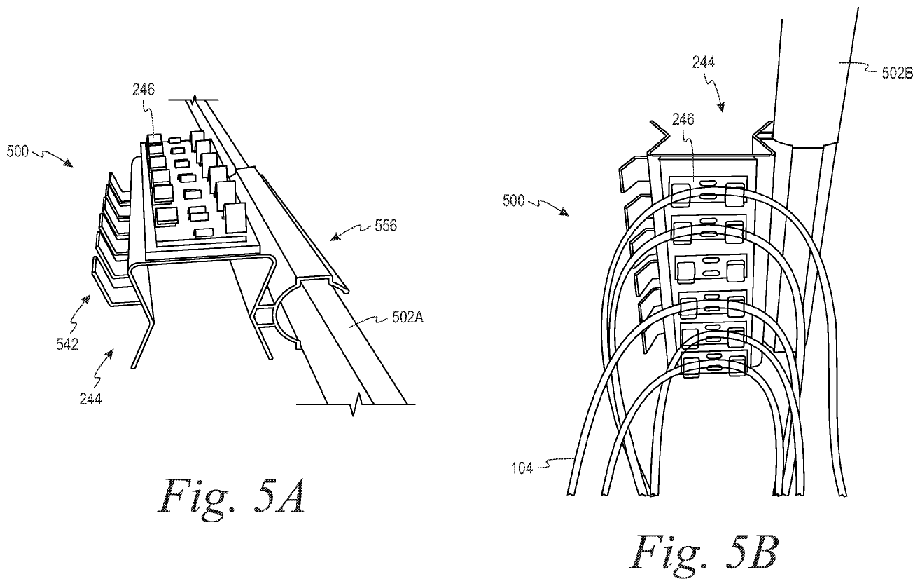

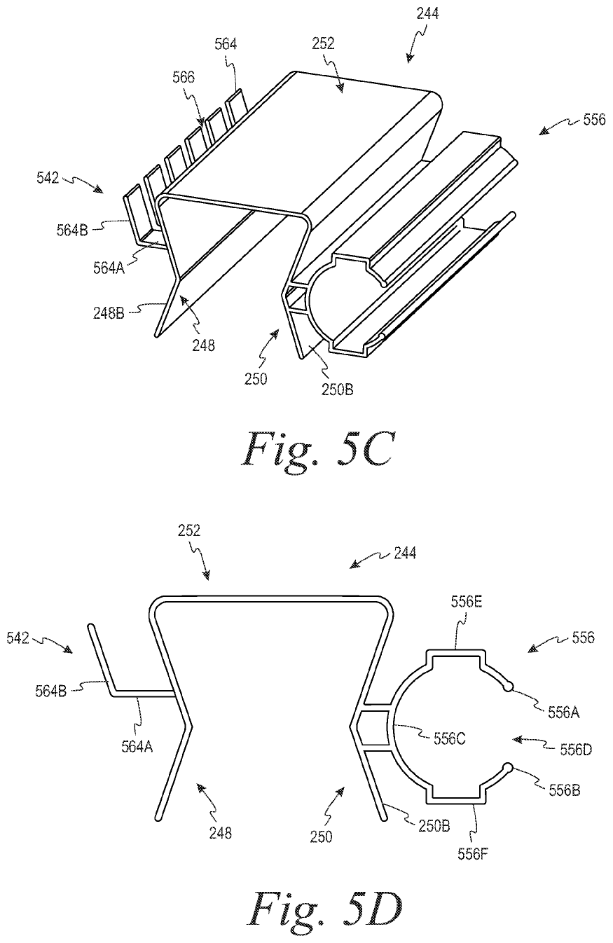

Referring now to FIGS. 5A-5D, a line management system 500 is shown according to another example. FIG. 5A depicts the line management system 500 coupled to a first support structure 502A, FIG. 5B depicts the line management system 500 coupled to a second support structure 502B and a plurality of fluid tubes 104, FIG. 5C depicts a perspective view of the main clip 544, a secondary clip 556, and a slack holder 542 for the line management system 500 of FIG. 5A. and FIG. 5D depicts a side view of the main clip 544, the secondary clip 556, and the slack holder 542 for the line management system 500 of FIG. 5A, according to an example.

The line management system 500 is substantially similar or identical to the line management system 300, except the secondary clip 556 and the slack holder 542 of the line management system 500 have alternative configurations according to other examples. As shown in FIGS. 5A-5D, the secondary clip 556 extends from the outer surface 250B of the second lateral wall 250 of the main clip 244. In this example, as shown in FIG. 5D, the secondary clip 556 includes a first end 556A, a second end 556B, an arc shaped segment 556C extending between the first end 556A and the second end 556B, and an aperture 556D between the first end 556A and the second end 556B. The arc shaped segment 556C can assist in coupling the secondary clip 556 to a support structure 102 having a rounded shape such, as for example, a support structure in the form of a pole.

The secondary clip 556 can also include a first rectangular segment 556E between the first end 556A and the arc shaped segment 556C, and a second rectangular segment 556F between the second end 556B and the arc shaped segment 556C. As shown in FIG. 5D, the first rectangular segment 556E can be aligned with the second rectangular segment 556F. This can help to additionally or alternatively couple the secondary clip 556 to a support structure 102 having a rectangular shape.

As shown in FIG. 5D, the secondary clip 556 can also include a second arc shaped segment 556G between the first rectangular segment 556E and the first end 556A, and a third arc shaped segment 556H between the second rectangular segment 556F and the second end 556B. The second arc shaped segment 556G and the third arc shaped segment 556H can further assist in coupling the secondary clip 556 to a support structure 102 that may be larger than a space defined between the first rectangular segment 556E and the second rectangular segment 556F.

As shown in FIGS. 5C-5D, the slack holder 542 can include a plurality of protrusions 564 extending from the outer surface 248B of the first lateral wall 248. The protrusions 564 each include (i) a lateral segment 564A that extends outwardly from the first lateral wall 248 (e.g., in a plane that is parallel to a plane of the end wall 252), and (ii) a vertical segment 564B that extends from the lateral segment 564A in a plane that is transverse to the plane of the end wall 252. As shown in FIG. 5C, the plurality of protrusions 564 are separated from each other such that a plurality of gaps 566 are defined between the protrusions 564. The protrusions 564 are configured to receive the fluid tubes 104 in the gaps 566 defined between the protrusions 564. In this arrangement, the fluid tubes 104 can be coupled to the slack holder 542 by wrapping the fluid tubes 104 around one or more of the protrusions 564 and through one or more of the gaps 566.

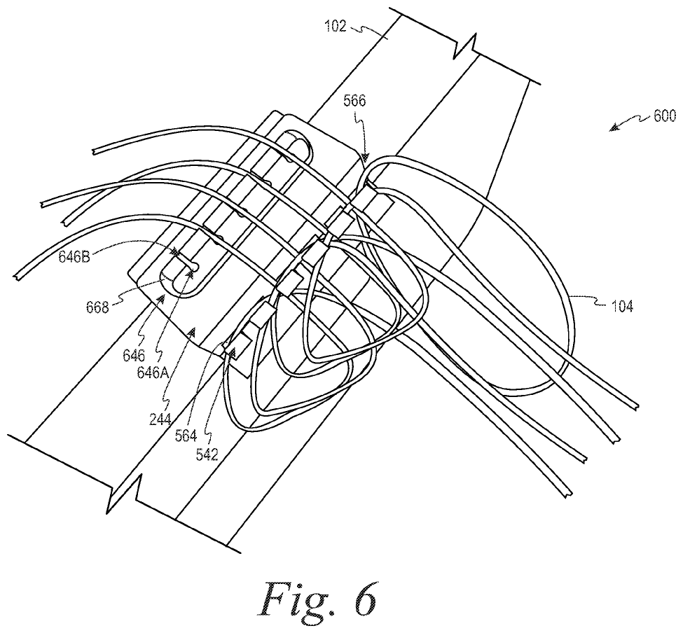

Referring now to FIG. 6, a perspective view of a line management system 600 coupled to a support structure 102 and a plurality of fluid tubes 104 is shown according to another example. The line management system 600 is substantially similar or identical to the line management system 500, except the line management system 600 includes a plurality of line retention structures 646 according to another implementation. As shown in FIG. 6, each line retention structure 646 one or more channels 646A formed in an elastically deformable substrate 668 coupled to the outer surface 250B of the end wall 252. The channels 646A can each have an aperture 646B for receiving the fluid tubes 104 into the channels 646A. The line retention structures 646 can be similar or identical to the first line retention structures 126, the second line retention structures 134, and/or the third line retention structures 136 described above.

As shown in FIG. 6, the fluid tubes 104 are coupled to the line retention structures 646 and a slack portion of the fluid tubes 104 is coupled to the slack holder 542. For instance, in FIG. 6, the slack portion of the fluid tubes 104 are wrapped around the protrusions 564 and through the gaps 566 between the protrusions 564.

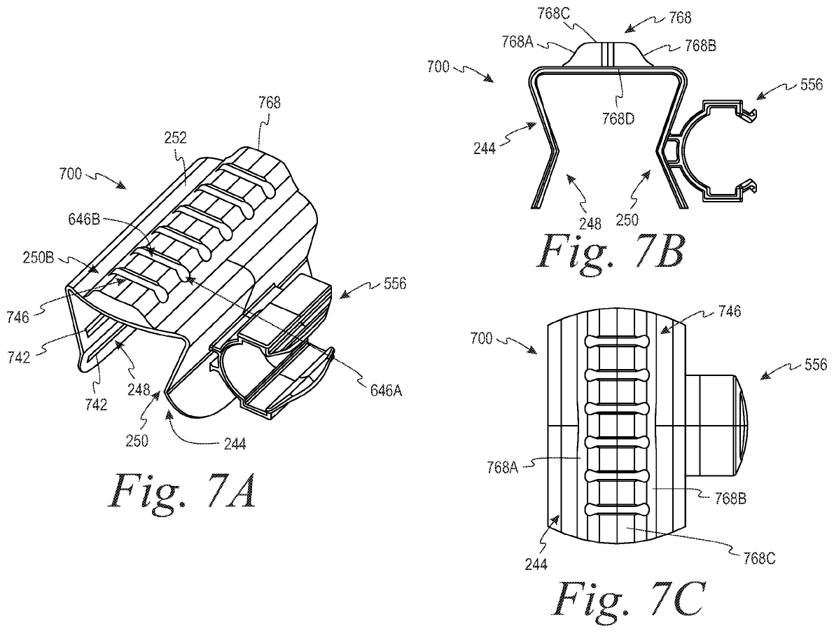

Referring now to FIGS. 7A-7C, a line management system 700 is shown according to another example. FIG. 7A depicts a perspective view the line management system 700, FIG. 7B depicts a side view of the line management system 700, and FIG. 7C depicts a top view of the line management system 700, according to an example. The line management system 700 is substantially similar or identical to the line management system 600 shown in FIG. 6, except the line management system 700 includes a plurality of line retention structures 746 according to another implementation and a slack holder 742 that is in the arrangement of the slack holder 442 shown in FIG. 4.

As shown in FIGS. 7A-7C, each line retention structure 746 one or more channels 746A formed in a substrate 768 coupled to the outer surface 250B of the end wall 252. The channels 646A can each have an aperture 646B for receiving the fluid tubes 104 into the channels 646A. The line retention structures 646 can be similar or identical to the first line retention structures 126, the second line retention structures 134, and/or the third line retention structures 136 described above.

As shown in FIGS. 7A-7C, the substrate 768 can have a first lateral side 768A and a second lateral side 768B. The first lateral side 768A can be nearer to the first lateral wall 248 of the main clip 244 than the second lateral wall 250 of the main clip 244, and the second lateral side 768B can be nearer to the second lateral wall 250 of the main clip 244 than the first lateral wall 248 of the main clip 244. The aperture 648B of each line retention structure 746 can be in a top surface 768C of the substrate 768. The channels 646A can each extend between the first lateral side 768A and the second lateral side 768B of the substrate 768. In one example, the substrate can be formed from an elastically deformable material.

As shown in FIG. 7B, the first lateral side 768A and the second lateral side 768B of the substrate 768 can have a beveled surface contour. For example, the top surface 768C of the substrate 768 can have a smaller width than a bottom surface 768D of the substrate 768. In this arrangement, along a dimension between the top surface 768C and the bottom surface 768D, the size of the substrate 768 can gradually increase. In some examples, the first lateral side 768A and the second lateral side 768B of the substrate 768 can have concave shapes. In other examples, the first lateral side 768A and the second lateral side 768B can have linear shapes. In implementations in which the first lateral side 768A and/or the second lateral side 768B have a beveled surface contour, the bevel can help to provide for easier coupling and decoupling of the fluid tubes 104 to the line retention structures 646 when tension is present on the fluid tubes 104.

Similar to the slack holder 442 described above, the slack holder 742 can couple to and retain one or more of the fluid tubes 104 while the fluid tubes 104 are also coupled to the line retention structures 746.

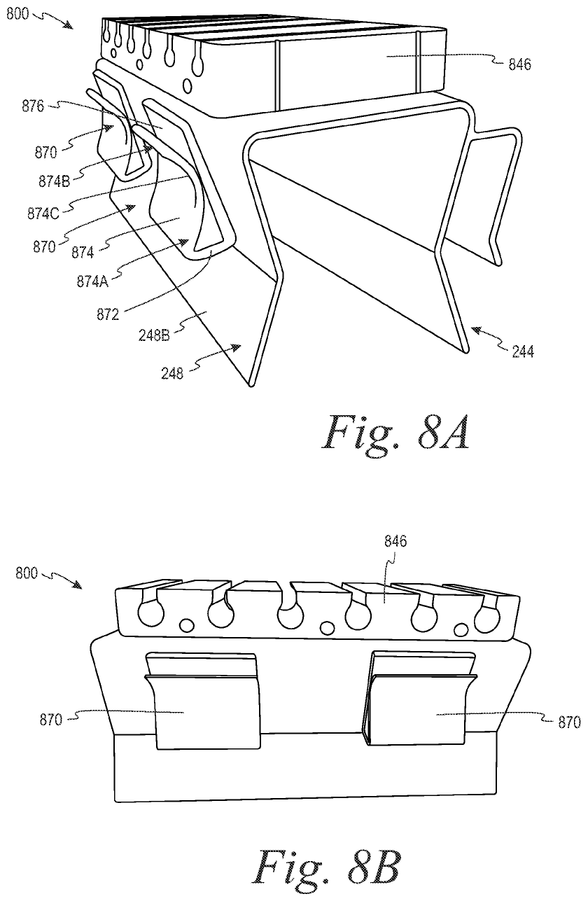

Referring now to FIGS. 8A-8B, a line management system 800 is shown according to another example. FIG. 8A depicts a perspective view the line management system 800, and FIG. 8B depicts a side view of the line management system 700. The line management system 800 is substantially similar or identical to the line management system 600 shown in FIGS. 3A-3B, except the line management system 800 includes one or more clips 870 and a plurality of line retention structures 846 in the form of the first line retention structures 126 described above.

The clip(s) 870 extend from the outer surface 248B of the first lateral wall 248. The clip(s) 870 can include a lateral segment 872 that extends outwardly from the first lateral wall 248, and a first vertical segment 874 that extends from the lateral segment 872 toward the end wall 252. The first vertical segment 874 can include a first portion 874A, a second portion 874B, and an intermediate point 874C between the first portion 874A and the second portion 874B. The first portion 874A can extend from the lateral segment 872 and define an acute angle with the lateral segment 872 such that the first portion 874A extends towards the first lateral wall 248. The second portion 874B can extend away from the first lateral wall 248 such that the first vertical segment 874 is closest to the first lateral wall 248 at the intermediate point 874C.

In FIGS. 8A-8B, the clip(s) 870 also include a second vertical segment 876 that extends along and is coupled to the first lateral wall 248. In other examples, the clip(s) 870 can omit the second vertical segment 876 and the lateral segment 872 can be directly coupled to or integrally formed with the first lateral wall 248 instead. In some examples, when the clip(s) 870 are in a natural resting state (e.g., when the first vertical segment 874 is not stretched away from the first lateral wall 248 and/or the first vertical segment 874 is not compressed toward the first lateral wall 248 by an external force), a distance between (i) the intermediate point 874C and (ii) the second vertical segment 876 and/or the first lateral wall 248 can be suitable to couple the main clip 244 to a fabric (e.g., a patient gown, a blanket, a bed sheet, etc.) without additional support. For instance, the first vertical segment 874 can be configured to contact the second vertical segment 876 and/or the first lateral wall 248 when the clip(s) 870 are in the natural resting state, and/or the distance can be less than a thickness of the fabric.

The clip(s) 870 can additionally or alternatively be configured to provide a slack holder. For instance, an excess portion of one or more the fluid tubes 104 can be formed into one or more loops of tubing and coupled to the main clip 244 by the clip(s) 870 in a manner similar to that described above and shown in FIG. 1F. This can further help to mitigate tangling and/or kinking.

The line management systems 100, 200, 300, 400, 500, 600, 700, 800 shown in FIGS. 1A-8B are example implementations of the concepts described herein. In other example implementations, any of the features of any of the illustrated examples can be combined with any of the feature of any other illustrated or described examples of the present application. For instance, example line management systems can include one or more features selected from among: (i) one or more of the line retention structures 126, 134, 136, 246, 646, 746, and/or 846, (ii) one or more of the attachment mechanism 138, (iii) the main clip 244 and/or 544, (iv) one or more of the secondary clip 356 and/or 556, (v) one or more of the slack holders 142, 442, 542, 742, and (vi) one or more of the clips 870.

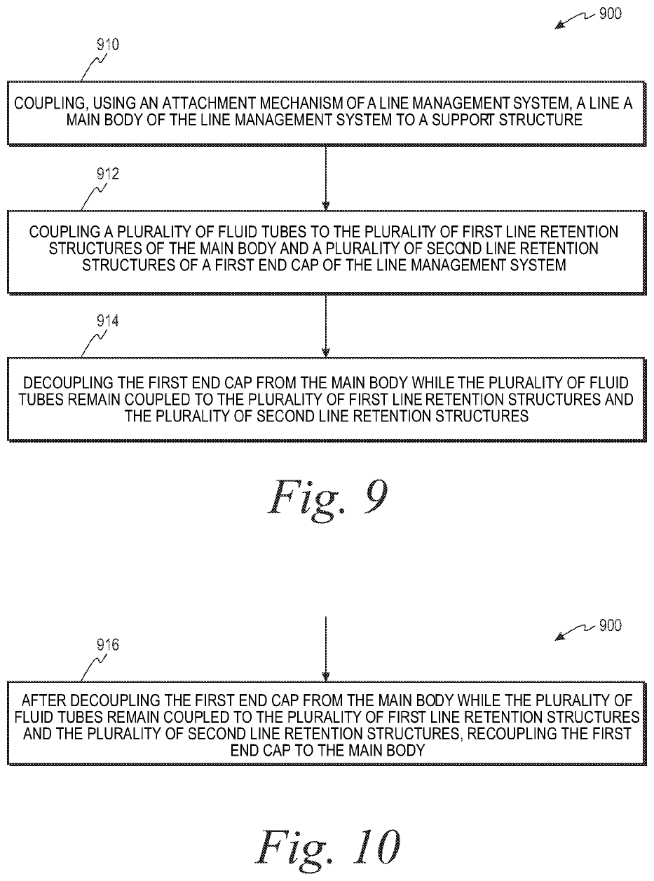

Referring now to FIG. 9, a flowchart of a process 900 of using a line management system is shown according to an example. At block 910, the process 900 includes coupling, using an attachment mechanism of a line management system, a line a main body of the line management system to a support structure. The main body includes (i) a first end surface, (ii) a second end surface opposite the first end surface, (iii) a first major surface extending between the first end surface and the second end surface, and (iv) a second major surface extending between the first end surface and the second end surface. The first major surface comprises a plurality of first line retention structures that each extend between the first end surface and the second end surface, and the second major surface is opposite the first major surface.

At block 912, the process 900 includes coupling a plurality of fluid tubes to the plurality of first line retention structures of the main body and a plurality of second line retention structures of a first end cap of the line management system. The first end cap is removably coupled to the first end surface. When the first end cap is coupled to the first end surface of the main body, each second line retention structure is aligned with a corresponding one of the plurality of first line retention structures such that the second line retention structure and the corresponding one of the plurality of first line retention structures simultaneously retain to a respective fluid tube of a plurality of fluid tubes.

At block 914, the process 900 also includes decoupling the first end cap from the main body while the plurality of fluid tubes remain coupled to the plurality of first line retention structures and the plurality of second line retention structures.

FIGS. 10-13 depict additional aspects of the process 900 according to further examples. As shown in FIG. 10, the process 900 can include, after decoupling the first end cap from the main body while the plurality of fluid tubes remain coupled to the plurality of first line retention structures and the plurality of second line retention structures at block 914, recoupling the first end cap to the main body at block 916.

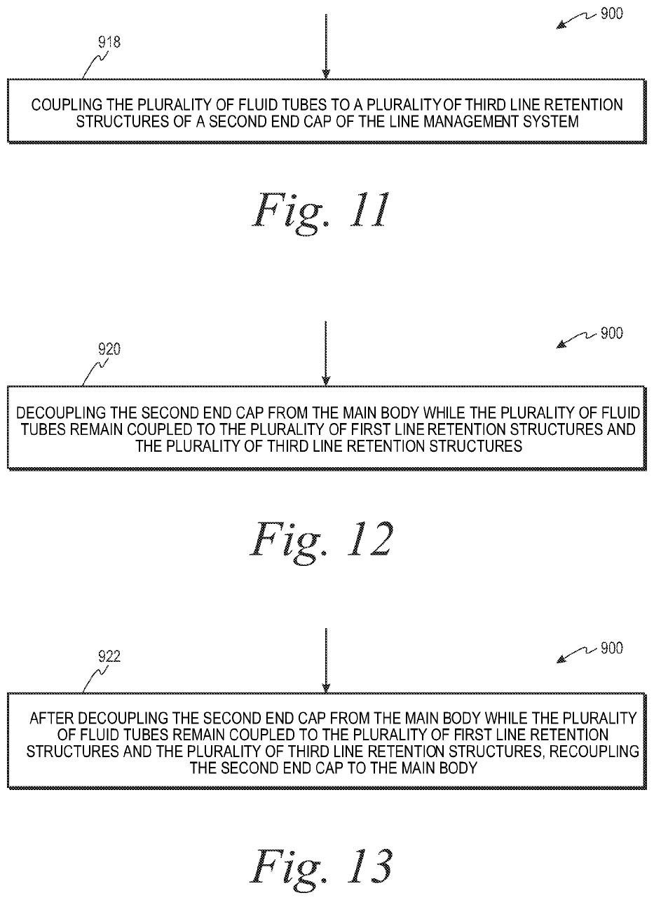

As shown in FIG. 11, the process 900 can also include coupling the plurality of fluid tubes to a plurality of third line retention structures of a second end cap of the line management system at block 918. The second end cap is removably coupled to the first end surface. When the second end cap is coupled to the second end surface of the main body, each third line retention structure is aligned with a corresponding one of the plurality of first line retention structures such that the third line retention structure and the corresponding one of the plurality of first line retention structures simultaneously retain to a respective fluid tube of the plurality of fluid tubes.

As shown in FIG. 12, the process 900 can also include decoupling the second end cap from the main body while the plurality of fluid tubes remain coupled to the plurality of first line retention structures and the plurality of third line retention structures at block 920.

As shown in FIG. 13, the process 900 can further include, after decoupling the second end cap from the main body while the plurality of fluid tubes remain coupled to the plurality of first line retention structures and the plurality of third line retention structures at block 920, recoupling the second end cap to the main body at block 922.

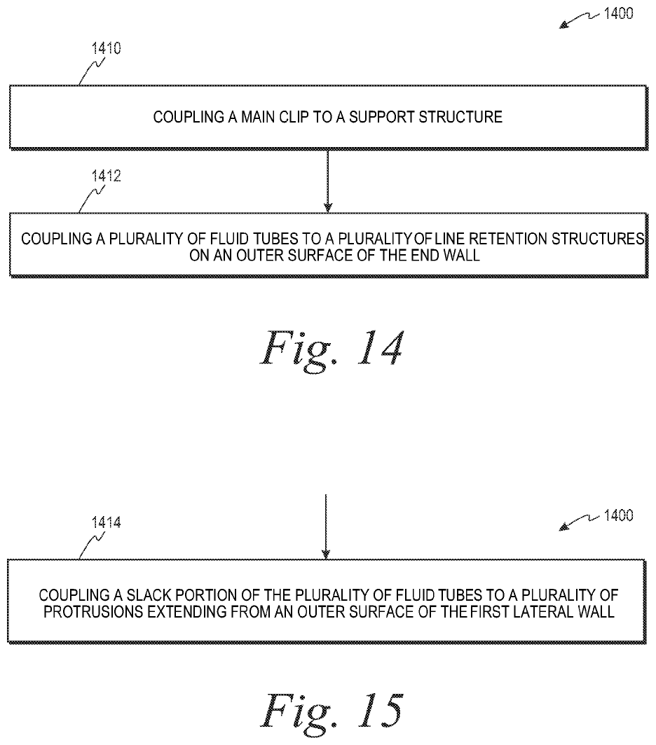

Referring now to FIG. 14, a flowchart of a process 1400 of using a line management system is shown according to another example. At block 1410, the process 1400 can include coupling a main clip to a support structure. The main clip includes a first lateral wall, a second lateral wall, and an end wall, wherein the first lateral wall extends from a first lateral end of the end wall and the second lateral wall extends from a second lateral end of the end wall such that an interior channel is defined between an inner surface of the first lateral wall, an inner surface of the second lateral wall, and an inner surface of the end wall.

At block 1412, the process 1400 includes coupling a plurality of fluid tubes to a plurality of line retention structures on an outer surface of the end wall.

FIGS. 15-18 depict additional aspects of the process 900 according to further examples. As shown in FIG. 15, the process 1400 can also include coupling a slack portion of the plurality of fluid tubes to a plurality of protrusions extending from an outer surface of the first lateral wall at block 1414. The plurality of protrusions are separated from each other such that a plurality of gaps are defined between the plurality of protrusions. The plurality of protrusions receive the plurality of fluid tubes in the plurality of gaps defined between the plurality of protrusions.

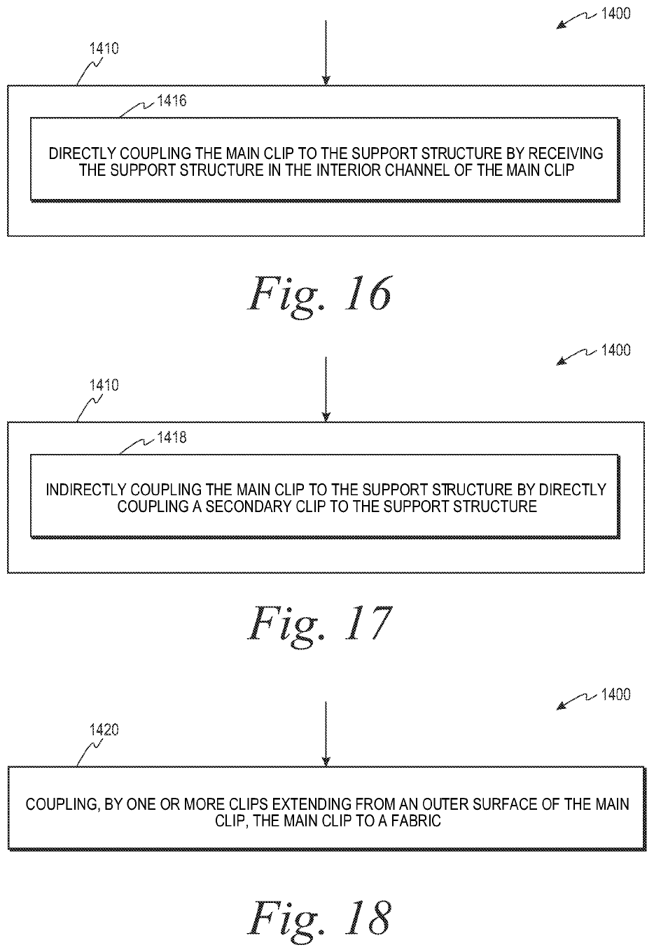

As shown in FIG. 16, coupling the main clip to the support structure at block 1410 can include directly coupling the main clip to the support structure by receiving the support structure in the interior channel of the main clip at block 1416.

As shown in FIG. 17, coupling the main clip to the support structure at block 1410 can include indirectly coupling the main clip to the support structure by directly coupling a secondary clip to the support structure at block 1418. The secondary clip extends from an outer surface of the second lateral wall.

As shown in FIG. 18, the process 1400 can also include coupling, by one or more clips extending from an outer surface of the main clip, the main clip to a fabric at block 1420.

The description of the different advantageous arrangements has been presented for purposes of illustration and description, and is not intended to be exhaustive or limited to the embodiments in the form disclosed. Many modifications and variations will be apparent to those of ordinary skill in the art. Further, different advantageous embodiments may describe different advantages as compared to other advantageous embodiments. The embodiment or embodiments selected are chosen and described in order to explain the principles of the embodiments, the practical application, and to enable others of ordinary skill in the art to understand the disclosure for various embodiments with various modifications as are suited to the particular use contemplated.

1. A line management system, comprising:
a main body comprising:
a first end surface,
a second end surface opposite the first end surface,
a first major surface extending between the first end surface and the second end surface, wherein the first major surface comprises a plurality of first line retention structures that each extend between the first end surface and the second end surface,
a second major surface extending between the first end surface and the second end surface, wherein the second major surface is opposite the first major surface,
an attachment mechanism configured to couple the main body to a support structure; and
a first end cap removably coupled to the first end surface, wherein the first end cap comprises a plurality of second line retention structures,
wherein, when the first end cap is coupled to the first end surface of the main body, each second line retention structure is aligned with a corresponding one of the plurality of first line retention structures such that the second line retention structure and the corresponding one of the plurality of first line retention structures are configured to simultaneously retain to a respective fluid tube of a plurality of fluid tubes.

2. The line management system of claim 1, wherein the main body has a longitudinal axis that extends between the first end surface and the second end surface,
wherein each first line retention structure comprises a channel defined by the main body and having a longitudinal axis that is parallel to the longitudinal axis of the main body, and
wherein the channel of each first line retention structure comprises an aperture that (i) extends along a length of the channel and (ii) has a width that is smaller than a diameter of the channel.

3. The line management system of claim 2, wherein at least a portion of the main body that defines the plurality of first line retention structures is formed from a elastically deformable material.

4. The line management system of claim 2, wherein each second line retention structure comprises a second channel defined by the first end cap, and
wherein the second channel of each second line retention structure comprises an aperture that (i) extends along a length of the channel and (ii) has a width that is smaller than a diameter of the second channel.

5. The line management system of claim 1, wherein (i) one of the first end surface or the first end cap comprises one or more protrusions, (ii) another of the first end surface or the first end cap comprises one or more receptacles, and (iii) the first end cap is removably coupled to the first end surface of the main body by a press-fit coupling between the one or more protrusions and the one or more receptacles.

6. The line management system of claim 1, wherein the attachment mechanism comprises at least one device selected from a group consisting of: one or more straps, one or more clips, and one or more adhesives.

7. The line management system of claim 6, wherein the attachment mechanism comprises the one or more straps, and
wherein the main body comprises one or more strap slots that are each configured to retain the one or more straps.

8. The line management system of claim 7, wherein the second major surface comprises the one or more strap slots.

9. The line management system of claim 7, wherein the main body comprises a first lateral surface extending between the first major surface, the second major surface, the first end surface, and the second end surface,
wherein the main body comprises a second lateral surface extending between the first major surface, the second major surface, the first end surface, and the second end surface,
wherein the first lateral surface and the second lateral surface comprise the one or more strap slots.

10. The line management system of claim 1, further comprising a second end cap removably coupled to the second end surface, wherein the second end cap comprises a plurality of third line retention structures,
wherein, when the second end cap is coupled to the second end surface of the main body, each third line retention structure is aligned with a corresponding one of the plurality of first line retention structures such that the third line retention structure and the corresponding one of the plurality of first line retention structures are configured to simultaneously retain to a respective fluid tube of the plurality of fluid tubes.

11. A line management system, comprising:
a main clip comprising a first lateral wall, a second lateral wall, and an end wall, wherein the first lateral wall extends from a first lateral end of the end wall and the second lateral wall extends from a second lateral end of the end wall such that an interior channel is defined between an inner surface of the first lateral wall, an inner surface of the second lateral wall, and an inner surface of the end wall; and
a plurality of line retention structures on an outer surface of the end wall.

12. The line management system of claim 11, wherein each line retention structure comprises one or more clips.

13. The line management system of claim 11, wherein each line retention structure comprises one or more channels formed in an elastically deformable substrate coupled to the outer surface of the end wall.

14. The line management system of claim 13, wherein the elastically deformable substrate comprises a beveled surface contour.

15. The line management system of claim 11, wherein the interior channel of the main clip comprises: (i) a first portion extending from the end wall to an intermediate point on the first lateral wall and the second lateral wall, and (ii) a second portion extending from the intermediate point on the first lateral wall and the second lateral wall to a free end of the first lateral wall and a free end of the second lateral wall, and
wherein the first portion has a different shape than the second portion.

16. The line management system of claim 15, wherein the first portion has a width that decreases in a direction from the end wall towards the intermediate point, and the second portion has a width that increases in a direction from the intermediate point toward the free end of the first lateral wall and the free end of the second lateral wall.

17. The line management system of claim 11, further comprising a plurality of protrusions extending from an outer surface of the first lateral wall, wherein the plurality of protrusions are separated from each other such that a plurality of gaps are defined between the plurality of protrusions, wherein the plurality of protrusions are configured to receive a plurality of fluid tubes in the plurality of gaps defined between the plurality of protrusions.

18. The line management system of claim 17, wherein the plurality of protrusions each comprise (i) a lateral segment that extends outwardly from the first lateral wall, and (ii) a vertical segment that extends from the lateral segment in a plane that is transverse to the plane of the end wall.

19. The line management system of claim 11, further comprising a secondary clip extending from an outer surface of the second lateral wall.

20. The line management system of claim 19, wherein the secondary clip comprises a first end, a second end, an arc shaped segment extending between the first end and the second end, and an aperture between the first end and the second end.

21. The line management system of claim 20, wherein the secondary clip further comprises:
a first rectangular segment between the first end and the arc shaped segment, and
a second rectangular segment between the second end and the arc shaped segment,
wherein the first rectangular segment is aligned with the second rectangular segment.

22. The line management system of claim 19, wherein the secondary clip comprises:
a lateral segment that extends from the second lateral wall, wherein the lateral segment has a first end at the second lateral wall and a second end opposite the first end; and
a vertical segment that extends from the second end of the lateral segment such that a second interior channel is defined between an outer surface of the second lateral wall, an inner surface of the lateral segment, and an inner surface of the vertical segment.

23. The line management system of claim 1, further comprising one or more clips that are configured to couple the main clip to a fabric without additional support.

24. The line management system of claim 1, wherein at least one of the first lateral wall or the second lateral wall comprises a plurality of strap slots that are configured to couple to a plurality of straps.

25-39. (canceled)
